(19) United States
(12) Patent Application Publication
(10) Pub. No.: US 2008/0220058 A1
(43) Pub. Date: Sep. 11, 2008

(54) Method and composition for improving hair growth

(76) Inventor: Robert McCarthy, Northport, NY (US)

Correspondence Address: Mitchell A. Stein, Esq.;STEIN LAW, P.C., Suite 4, 24 Woodbine Avenue, Northport, NY, 11768, US

(21) Appl. No.: 11/716,224

(22) Filed: Mar. 9, 2007

(51) Int. Cl. A61K 9/48; A61K 31/40; A61K 31/66; A61K 36/00

(52) U.S. Cl. 424/456; 424/725; 514/410; 514/78

(57) ABSTRACT

A method and composition for improving human hair, having a composition of neutraceuticals that include Eclipta, chlorophyll, and an absorption enhancer, in which the absorption enhancer is selected from the group consisting of bioperine and phosphatidyl choline and Eclipta is included in an amount between 0.1 and 60 parts admixed by weight of the composition. The method and composition can be administered: (a) sublingually, having a solute, preferably ethanol, and preferably in the range between 0.1% and 30% by volume; (b) orally by encapsulation in a gelatinous capsule; and/or (c) by chewable mixture having a gum material and/or a flavor.

### FIELD OF THE INVENTION

The present invention relates to the field of neutraceutical administration for the restoration of thinning hair and promotion of hair growth, prevention of graying hair, and darkening of hair via the stimulating production of hair follicles using sublingual, encapsulated, and chewable compositions comprising, inter alia, Eclipta alba and/or Eclipta prostrada in new, syngertistic combinations with other ingredients for enhancement.

### BACKGROUND OF TH INVENTION

The average human head has approximately 100,000 follicles which can each grow approximately 20 individual hairs over an average lifetime. However, people do not retain all hair grown throughout their lifetime. Instead, humans lose hair on a daily basis. Indeed, the average person loses approximately 100 hairs per day, perhaps without even realizing it.

Typically, hair regrowth follows hair loss. However, due to a variety of known and unknown reasons, hair either fails to regrow as strong and healthy as it was prior to hair loss, or fails to regrow at all. Although one's failure to regrow hair as strong and healthy as it was prior to hair loss, or failure to regrow at all is genetically determined, individuals can begin to experience such symptoms as early as puberty. When hair fails to regrow as strong and healthy as it was prior to hair loss, any hair that is regrown appears to be thin (a result of a process known as follicular miniaturization). With follicular miniaturization, hair shaft width is progressively decreased until scalp hair resembles fragile vellus hair or “peach fuzz” or else becomes non-existent. The follicles of individuals with follicular miniaturization decrease in diameter to the point that they cannot replace lost hairs.

When hair fails to regrow, and continues to thin, baldness, also known as alopecia, occurs. Baldness can occur in varying degrees, such as androgenic alopecia (male pattern baldness), alopecia areata (which involves some hair loss), alopecia totalis (which involves loss of all head hair), and alopecia universalis (which involves loss of all head and body hair). Androgenic alopecia alone affects 40 million male adults in the United States alone. Indeed, approximately 25% of men begin to experience some degree of hair loss (and non-regrowth) by the age of 30. Alopecia areata, on the other hand, affects approximately 4 million Americans. The various forms of alopecia are generally considered to be auto-immune conditions in which white blood cells attack the hair follicles, which leads to hair loss.

The actual cause of alopecia may actually be from a number of factors including: poor nutrition (including various gastrointestinal problems), improper grooming, weight loss, pregnancy, menopause, stress, medication, thyroid dysfunction, scalp disorders, or over-processing the hair (e.g. coloring, ironing, etc.). However, the incidence of alopecia is not affected by environmental factors. Rather, the incidence of alopecia appears to be directly related to genetic background. However, there are a number of contributing factors. For example, emotional stress has been shown to accelerate baldness in genetically susceptible individuals.

Hair loss typically has psychological implications. Balding can often make an individual look or feel older than their actual age. Indeed, some individuals deal with balding or baldness very well, while other individuals have a hard time coping with balding or baldness. Those individuals who experience difficulty in coping with their baldness often have severe problems relating to, among other things, loss of self-esteem, anxiety, depression, and social phobia.

The causes of hair growth, and hence hair loss, are believed to be, at least in part, metabolic in origin. For example, dihydrotestosterone (DHT) is active metabolite of the hormone testosterone that is believed to be an important contributor to, among other things, facial and body hair growth in males. Although, the actual mechanism by which DHT affects facial and body hair growth is not yet understood, DHT is thought to be a contributing factor in most cases of androgenic alopecia (male pattern baldness). As such, 5α-reductase inhibitors, which inhibit DHT production, are commonly used to treat male pattern baldness. Alternative treatments used to inhibit DHT include saw palmetto berry extracts.

There are many products on the market today that claim to reverse baldness and/or regrow hair by inhibiting DHT production. For example, finasteride (marketed by Merck as “Propecia” and/or “Proscar”) is marketed for the treatment of male pattern baldness in men. Finasteride is a 4-aza steroid compound that acts by inhibiting 5α-reductase, the enzyme that converts testosterone to DHT. However, finasteride is known to have certain sexual side effects such as: less desire for sex, difficulty in achieving an erection, or a decrease in the amount of semen.

Other known 5α-reductase inhibitors, which may be used to stimulate hair growth by inhibiting DHT production are: turosteride, LY-191704, MK-306, ketoconazole, and dutasteride. It should be recognized that over time use of 5α-reductase inhibitors will result in a loss of effectiveness. Indeed, the effectiveness of the many 5α-reductase inhibitors may be limited to five (5) years.

Alternative treatments used to inhibit DHT include saw palmetto berry (Serenoa repens) extracts.

Another product currently on the market that claims to reverse baldness and/or regrow hair, is minoxidil (marketed by Upjohn Corporation as “Rogaine”). Minoxidil is a vasodilator that inhibits nitric oxide production to stimulate hair growth and treat hair loss. However, minoxidil is known to have side effects such as: itchy scalp, acne, headaches and/or lightheadedness, very low blood pressure, irregular or fast heart beat, blurred vision, and chest pain. Moreover, some individuals may experience allergic reactions to minoxidil or one of its non-active ingredients.

Recent studies have shown that treatment using 5α-reductase inhibitors (such as finasteride) and vasodilators (such as minoxidil) are generally ineffective at treating extreme cases of hair loss. Moreover, known treatments, such as finasteride and minoxidil have shown greater success in preventing the falling out of healthy hairs than regrowing hair in follicles that have already become dormant.

Other methods used to deal with baldness include use of a hairpiece or hair transplantations. However, the aforementioned products and methods have been met with little or no success. Moreover, because products, such as a hairpiece do not accurately mimic natural human hair, they often fail to counteract the psychological implications of balding. Indeed, a hairpiece does little more than conceal hair loss.

Another method of reversing hair loss is hair transplantation, whereby active (non-dormant) hair-producing follicles are taken from the back and sides of the head and injected into bald or thinning areas on the top and front of the head.

Unlike chemical-based products such as 5α-reductase inhibitors (such as finasteride) and vasodilators (such as minoxidil), herbal remedies are generally compatible with the human body, safer and extremely effective. Herbal remedies to combat hair loss are virtually unknown in the United States, but have been used in Asian cultures for thousands of years. For example, a product known as “Eclipta-Raj Hair Oil,” which contains sesame seed oil, eclipta erecta, emblica offici nalis, terminalia belerica, and Hydrocotyle asiatica claims to restore hair growth. According to Ayurevda, the company that markets “Eclipta-Raj Hair Oil,” the product promotes hair growth by “nourishing the hair follicles from the inside out.” An individual that wishes to use “Eclipta-Raj Hair Oil” to restore hair growth cannot consume it orally, instead they must apply the product prior to shampooing. Heretofore unknown is an herbal composition available in sublingual, encapsulated, and chewable forms.

It is thus an object of the instant invention to provide an herbal composition for sublingual, encapsulated, and chewable administration that minimizes thinning hair, stimulates hair growth, prevents graying hair, and darkens hair, using safe, syngertistic herbal components.

### SUMMARY OF THE INVENTION

The various features of novelty which characterize the invention are pointed out with particularity in the claims annexed to and forming a part of the disclosure. For a better understanding of the invention, its operating advantages, and specific objects attained by its use, reference should be had to the drawings and descriptive matter in which there are illustrated and described preferred embodiments of the invention.

The present invention comprises a method and composition system for improving human hair, having a composition of neutraceuticals that include Eclipta, chlorophyll, and an absorption enhancer, in which the absorption enhancer is selected from the group consisting of bioperine and phosphatidyl choline and Eclipta is included in an amount between 0.1 and 60 parts admixed by weight of the composition. The method and composition can be administered: (a) subingually, having a solute, preferably ethanol, and preferably in the range between 0.1% and 30% by volume; (b) orally by encapsulation in a gelatinous capsule; and/or (c) by chewable mixture having a gum material and/or a flavor.

As discussed hereinabove, treatments for alopecia have enjoyed varying success. Indeed, the success of the treatment is believed to be inversely proportional to the amount of hair lost.

Eclipta alba (syn. Eclipta prostrada, Eclipta erecta, also known as “Bringraj,” “Trailing Eclipta,” “False Daisy,” “Kalkeshi,” “Keshrag,” “Bhangra,” “Maka,” “Cotula,” “Han Lin Cao,” “takasaburou,” “yerba de tago,” and “congo lanna”) is an herb belonging to the family Asteraceae that grows abundantly in humid climates. Eclipta alba is a creeping and moisture-loving herb which bears small solitary white hermaphroditic flowers (6-8 mm in diameter) atop a long stalk. Eclipta alba is considered a weed in most places in the world, as it is native to tropical and subtropical regions. Indeed, there has been much effort to develop herbicides to prevent the prolific growth of Eclipta alba in places where its growth is not desired. For example, see U.S. Pat. No. 5,622,913 to Cross, et al., disclosing heteropyridine compounds for controlling a wide variety of annual and perennial plant species, including Eclipta alba; U.S. Pat. No. 5,017,215 to Ackerson, et al., disclosing a mixture of herbicidal compounds for controlling the growth of undesired vegetation, including Eclipta alba; U.S. Pat. No. 4,557,750 to Imai, et al., disclosing sulfinamide derivatives for preparing an herbicide to combat, among other things, Eclipta alba.

Eclipta alba can be cooked and used as a vegetable. Tanaka, T., Tanaka'Cyclopaedia of Edible Plants of the World (Keigaku Publishing 1976); Kunkel, G., Plants for Human Consumption (Koeltz Scientific Books 1984).

Eclipta alba has also been used in herbal formulations prescribed for: liver ailments (J. M. Widespread. Capparidaceae. Flora Malesiana Series I. 6: 61-105 (1972); treatment of hepatitis (Singapore science centre resource page. Available at: http://www.science.edu.sg/ssc/wildflowers/catswhiskerfamily.jsp); wound healing (K. R. Kiritikar, B. D. Basu, Indian Medicinal Plants, (Lalit Mohan Basu, Deharadun, India, 1991) p. 181.)); as an antihyperglycemic (B. Waterhouse, A. Mitchell, Northern Australia Quarantine Strategy Weeds Target List, (AQIS Miscellaneous Publication, 1998) p. 29); as an anti-hemmorrhagic (Kosuge T, Yokota M, Yoshida M, Ochiai A. 1981. Studies on antihemorrhagic principles in the crude drugs for hemostatics. Yakugaku Zasshi 101:501-503); and even as an anti-venom to treat snakebites (Mors, W. B., Nascimento, M. C., Parente, J. P., Silva, M. H., Melo, P. A. and Suarez-Kurtz, G. 1989 Neutralization of Lethal and Myotoxic Activities of South American Rattlesnakes Venom by Extracts and Constituents of the Plant Eclipta prostata (Asteraceae). Toxicon., 27, 1003-1009).

Eclipta alba has been used in Asian cultures for generations for a whole host of purposes including to: prevent aging, maintain and rejuvenate hair, teeth, bones, memory, sight, and hearing, rejuvenate the kidneys, remove graying, cure balding, make hair darker, promote sleep, improve complexion, treat hepatitis, treat skin disorders, relieve burning urine, relieve headache, alleviate excess mucus, remove worms, treat earaches, treat uterine hemorrhaging, and to remove gray hair and balding. While Eclipta alba extracts have been used in traditional Chinese medicine as well as by Middle-eastern Arurvedic doctors for thousands of years to prevent hair loss, heretofore unknown is a synergistic herbal composition available in sublingual, encapsulated, and chewable forms for, among other things, minimizing hair loss. Although the chemical component of Eclipta alba which promotes hair growth has not yet been identified, it is believed that Eclipta alba's ability to promote hair growth stems from its bioactive steroidal alkoids.

The components of the instant invention are added to ground Eclipta alba to promote healthy hair. It is a synergistic composition; the components are critical in order to provide the benefits discussed hereinabove and to prevent unwanted side effects. The components are the result of numerous experiments and data accumulation in concluding efficacy.

Chlorophyll is a green photosynthetic pigment found in most plants. Chlorophyll contains more light energy than any other element. Recent studies have shown that human blood, which carries oxygen to all cells in the human body, is practically identical to chlorophyll on the molecular level. As a result, chlorophyll has numerous benefits, including: aiding in rebuilding the bloodstream, refining tissue, detoxification, increasing energy, neutralizing toxins in the body and preventing bacterial growth and development. In accordance with the instant invention, it has been found that the addition of chlorophyll to ground Eclipta enhances both the efficacy of the final product for healthy hair, as well as increasing shelf life.

Bioperine® is an extract of Piper nigrum or Piper longum containing approximately 95% piperine. Sabinsa Corporation, the company that markets Bioperine, claims that Biopene “significantly enhances the bioavailability of various supplement nutrients through increased absorption.” (http://www.bioperine.com). Heretofore, Bioperine has only been used to improve gastrointestinal absorption. See, for example, U.S. Pat. No. 5,536,506, U.S. Pat. No. 5,744,161, U.S. Pat. No. 5,972,382, and U.S. Pat. No. 6,054,585.

Phosphatidylcholine is a polar phopholipid important for normal cellular membrane composition and repair, which is basic to all biological processes. Phosphatidylcholine obtained from plants is considered to be generally regarded as safe.

Either bioperine or phosphatidylcholine are considered appropriate carriers to enhance delivery of the composition. However, it should be appreciated that where the encapsulated formulation is utilized, other elements may be added to render the composition lipophyllic, and hence easily absorbable in the human gastrointestinal system without premature enzymatic breakdown.

The sublingual, encapsulated, and chewable compositions disclosed and claimed herein provide physiological benefits including, restoring hair growth, darkening hair color and/or preventing premature graying of hair, improving hair luster, preventing and curing alopecia, preventing hair loss due to mental strain or illness, preventing hair from thinning, strengthening hair, and preventing hair loss.

After detailed study and analysis, the instant invention combines critical ingredients that have been discovered to work synergistically to restore hair growth, darken hair color and/or prevent premature graying of hair, improve hair luster, prevent and cure alopecia, prevent hair loss due to mental strain or illness, prevent hair from thinning, strengthen hair, and prevent hair loss without causing any negative side effects. Eclipta alba, a herb with many benefits, with this composition has been shown to promote healthy hair in all the ways discussed above.

It is therefore an object of the instant invention to provide sublingual, encapsulated, and chewable compositions to restore hair growth.

It is a further object of the instant invention to provide sublingual, encapsulated, and chewable compositions to darken hair color and/or prevent premature graying of hair.

It is a further object of the instant invention to provide sublingual, encapsulated, and chewable compositions to improve hair luster.

It is a further object of the instant invention to provide sublingual, encapsulated, and chewable compositions to prevent and cure alopecia.

It is a further object of the instant invention to provide sublingual, encapsulated, and chewable compositions to prevent hair loss due to mental strain or illness.

It is a further object of the instant invention to provide sublingual, encapsulated, and chewable compositions to prevent hair from thinning.

It is a further object of the instant invention to provide sublingual, encapsulated, and chewable compositions to strengthen hair.

It is a further object of the instant invention to provide sublingual, encapsulated, and chewable compositions to prevent hair loss.

It is a further object of the instant invention to provide sublingual, encapsulated, and chewable compositions to promote overall health.

It is a further object of the instant invention to provide sublingual, encapsulated, and chewable compositions that will not cause any negative side effects.

It is a further object of the instant invention to provide sublingual, encapsulated, and chewable compositions that is all natural.

It is a further object of the instant invention to provide sublingual, encapsulated, and chewable compositions that lacks preservatives.

The foregoing objects and other objects of the invention are achieved through a method of combining Eclipta alba with chlorophyll and bioperine and/or phosphatidylcholine.

It is a further object of the instant invention to provide sublingual, encapsulated, and chewable compositions to promote overall health. Other features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims.

### BRIEF DESCRIPTION OF THE DRAWINGS

In the drawings, wherein similar reference characters denote similar elements through the several views:

FIG. 1 is an overview of the instant invention showing the preferred embodiments, method and composition.

### DETAILED DESCRIPTION OF THE PREFERRED EMBODIMENTS

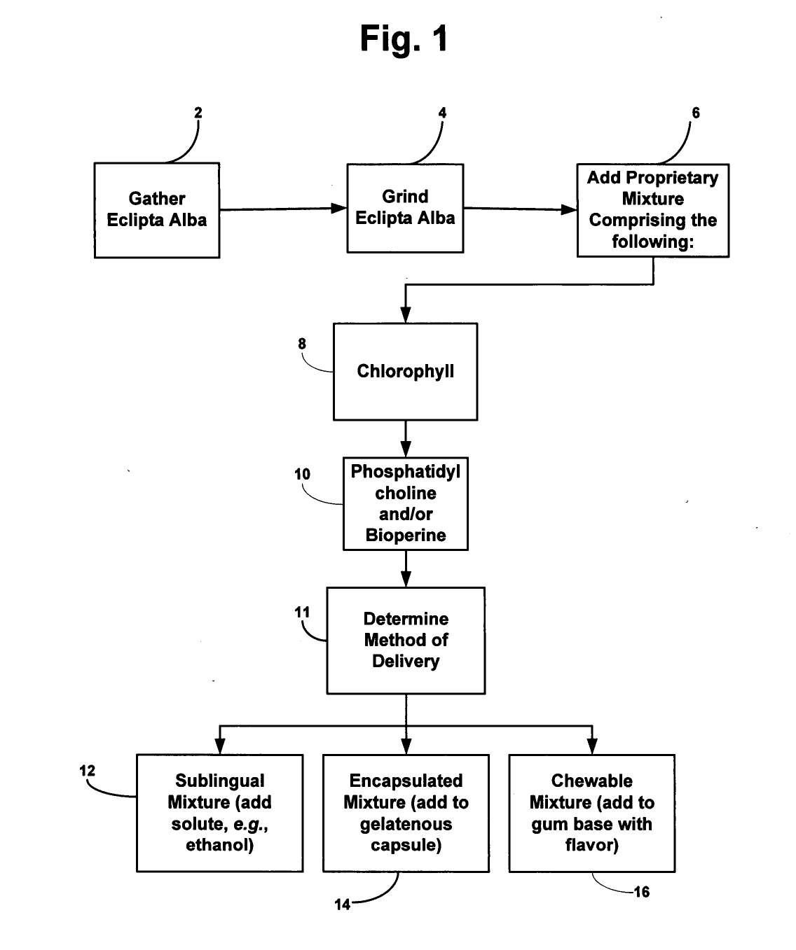

In accordance with the subject invention, FIG. 1 shows an overall view of the various mechanisms to combine Eclipta alba (sometimes referred to herein as “Eclipta”) with chlorophyll and bioperine and/or phosphatidylcholine, as well as the proprietary composition(s).

In particular, Eclipta is gathered at step 2, it been understood that the gathering can be from fresh, dried, or processed Eclipta alba plants, depending on the desire of the end-user.

Under a preferred embodiment of the invention, Eclipta is gathered at step 2, and the proprietary mixture added at step 6, it being understood that the plant in either fresh, dried, or processed form is ground before use at step 4.

The proprietary mixture is added at step 6 in an amount between 0.1 to 60 parts by weight of the final composition. As shown in FIG. 1, it has been determined that at least two key ingredients in addition to Eclipta are critical, in order for the sublingual, encapsulated, or chewable composition to achieve the efficacy sought. In particular, at step 8, chlorophyll is added. Likewise, at step 10 either phosphatidylcholine and/or bioperine is added.

Thereafter, under the method, a determination is made at step 11 of the method of delivery. Where delivery is sublingual, the mixture is created at step 12 by adding a solute, e.g., ethanol, and preferably in the range of 0.1% to 30% by volume. Where the method is oral administration, the composition is encapsulated by adding to a gelatinous capsule. Where the method is a chewable mixture, the composition is added to a gum based with flavor. It should be appreciated that one of ordinary skill in the art can determine from the subject disclosure the various quantities, mechanisms and materials to achieve the goal without deviating from the spirit or intent of the subject invention.

While there have been shown, described and pointed out fundamental novel features of the invention as applied to preferred embodiments thereof, it will be understood that various omissions and substitutions and changes in the form and details of the device illustrated and in its operation may be made by those skilled in the art without departing from the spirit of the invention. It is the intention, therefore, to be limited only as indicated by the scope of the claims appended hereto.

I claim:

1. A method for improving human hair, comprising administering to the human a composition of neutraceuticals comprising Eclipta, chlorophyll, and an absorption enhancer.

2. The method of claim 1, wherein the absorption enhancer is selected from the group consisting of bioperine and phosphatidyl choline.

3. The method of claim 1, wherein said Eclipta comprises between 0.1 and 60 parts admixed by weight of the composition.

4. The method of claim 1, wherein said administration is sublingual, and said composition further comprises a solute in which said neutraceuticals are soluble.

5. The method of claim 3, wherein said solute is ethanol.

6. The method of claim 4, wherein said solute comprises between 0.1% and 30% by volume.

7. The method of claim 1, wherein said administration is encapsulated.

8. The method of claim 7, wherein said encapsulation utilizes a gelatinous capsule.

9. The method of claim 1, wherein said administration is by chewable mixture.

10. The method of claim 9, wherein said chewable mixture further comprises a gum material.

11. The method of claim 10, wherein said chewable mixture further comprises a flavor.

12. A composition for improving human hair, comprising:
(a) Eclipta;
(b) chlorophyll; and
(c) an absorption enhancer.

13. The composition of claim 12, wherein the absorption enhancer is selected from the group consisting bioperine and phosphatidyl choline.

14. The composition of claim 12, wherein said Eclipta comprises between 0.1 and 60 parts admixed by weight of the composition.

15. The composition of claim 12, wherein said composition is delivered, selected from the delivery mechanisms consisting of sublingual, encapsulation and chewable mixture.

16. The composition of claim 15, wherein the delivery mechanism is sublingual, and the composition further comprises a solute.

17. The composition of claim 16, wherein the solute is ethanol

18. The composition of claim 16, wherein said solute comprises between 0.1% and 30% by volume.

19. The composition of claim 15, wherein the delivery mechanism is encapsulation in a gelatinous capsule.

20. The composition of claim 15, wherein the delivery mechanism is a chewable mixture comprising a gum base and a flavor.
